(19) United States
(12) Patent Application Publication
(10) Pub. No.: US 2025/0052446 A1
(43) Pub. Date: Feb. 13, 2025

(54) DISINFECTION / STERILIZATION DEVICE FOR AIR CONDITIONING SYSTEMS

(71) Applicant: Mattia NUTI, La Spezia (IT)

(72) Inventor: Mattia NUTI, La Spezia (IT)

(21) Appl. No.: 18/719,313

(22) PCT Filed: Dec. 12, 2022
(86) PCT No.: PCT/IT2022/050323

§ 371 Date: Jun. 13, 2024

(30) Foreign Application Priority Data
Dec. 13, 2021 (IT) 102021000031190

(51) Int. Cl. F24F 13/20; F24F 1/0071; F24F 8/22

(52) U.S. Cl. CPC F24F 13/20; F24F 1/0071; F24F 8/22

(57) ABSTRACT

Air disinfection/sterilization device and group is described conveniently usable for the disinfection/sterilization of air conditioning systems, comprising: an air conditioner comprising an internal unit comprising an outlet mouth, crossed by air in output from the internal unit and an inlet mouth crossed by air in input from the internal unit; a disinfection/sterilization device comprising: a boxed longitudinal frame, extending longitudinally along the length of the outlet mouth or the inlet mouth of the air conditioner; disinfection/sterilization means, adapted to purify air in output from the outlet mouth and/or in input from the inlet mouth; the disinfection/sterilization means comprise at least one IIV-C type LED; means for coupling the boxed longitudinal frame to the air conditioner and means for coupling the boxed longitudinal frame to the air conditioning system; the means for coupling the boxed longitudinal frame to the air conditioner comprising gluing and adhesive tapes used to anchor the boxed longitudinal frame near the outlet mouth or the inlet mouth of the air conditioner and, further, to an air conditioning duct which is afferent to air intake and/or exhaust vents.

### TECHNICAL FIELD

The present patent application for industrial invention relates to a disinfection/sterilization device for air conditioning systems.

In the present patent, the term air conditioning systems is intended to include air conditioners, split and ducted air conditioning systems.

In particular, the disinfection/sterilization device can be applied to split air diffusers but also in ducted air conditioning systems.

The reference field in which the present invention has been conceived is that of thermal machines, in particular that related to air conditioners and other air conditioning systems.

### BACKGROUND ART

An air conditioner or split is designed to be installed in public and/or private enclosed places in order to heat and/or cool said place, in which the air conditioner is positioned.

The air conditioner is intended to be activated when it is desired to provide hot or cold air to the place where it is installed.

The use of the air conditioner is in a sense conditioned by the outside temperature, which obviously depends on the current season and the geographical location in which it is located; as everyone knows during the summer season the air conditioner is activated so as to provide cold air, while during the winter season, it is activated so as to provide hot air.

The activation of the air conditioner is thus aimed at changing the temperature within an environment, so as to offer comfort to those who are located in said environment.

Commercial air conditioners do not allow the disinfection/sterilization of the air they emit; in other words, they do not comprise disinfection/sterilization devices therein, thus if on the one hand the air conditioner is a device which allows the temperature of a closed environment to be brought to a comfortable temperature, on the other hand it does not allow to sterilize the air in said environment.

At a time when there is a need not only to change the temperature of the air contained in an environment, but also to perform a purification/sanitation of the air itself, we are currently forced to purchase a separate purifying device.

Alternatively, in order to avoid purchasing a further device, subject to wear, maintenance and specific assistance, some companies in the field of air conditioners and spare parts propose modifications to the traditional air conditioners through disinfection/sterilization filters.

More precisely, said modifications envisage replacing the original filter, installed inside the air conditioner, with a disinfection/sterilization filter.

Obviously, to perform said modification, it is necessary to find a disinfection/sterilization filter compatible with the air conditioner on the market, in terms of size and operating mode, and said disinfection/sterilization filters are not always produced.

In the state of the art, further modifications to conditioning systems for purification/sanitization are known, such as the use of air deflectors to divert impurities as in the case of patent CN204943825U or deflectors provided with a coating of purifying material as in patent CN106123286A and finally deflectors provided with disinfection/sterilization systems as in patent CN211926075U.

Furthermore, the use of ejection grids made of photocatalytic material is known as in patent EP3835098A1 or purifying grids based on photoelectric purification techniques as in patent CN213955563U as a substitute.

In any case, the aforementioned interventions for the modification, addition or replacement of elements are impractical and inefficient because often it is not possible to process the entire flow of air emitted and sometimes even dangerous for human health with certainty. In addition, said modifications for existing systems must be entrusted to a specialized professional, who must go to the building in which an air conditioning system is installed, disassemble it, replace the components, while avoiding damaging the pre-existing structure.

### OBJECTS OF THE INVENTION

In this regard, it is an object of the present invention to devise an air disinfection/sterilization group, comprising an air conditioner, or any other type of air conditioning system, which is capable of alternatively providing hot purified/disinfected/sterilized air or cold purified/disinfected/sterilized air.

A further object of the present invention is to devise a disinfection/sterilization device which is versatile and adaptable to any type of air conditioner; in doing so the owner of an air conditioner, interested in the purification/disinfection/sterilization of the air in which the air conditioner is located, would not be forced to modify his own air conditioner or, worse still, replace it with a different one having the desired functions air purification/disinfection/sterilization functions.

### DISCLOSURE OF THE INVENTION

These objects are achieved in accordance with the invention by means of a disinfection/sterilization device capable of sterilizing/disinfecting/purifying the air immediately in output from the air conditioning systems and operating according to the features listed in the independent appended claim 1. Advantageous embodiments appear from the dependent claims. The disinfection/sterilization group according to the invention is defined by claim 1. The disinfection/sterilization device of the aforementioned group is defined by claim 9.

### BRIEF DESCRIPTION OF THE ACCOMPANYING DRAWINGS

For greater explanatory clarity, the description of the disinfection/sterilization group according to the invention continues with reference to the accompanying drawings, having only an illustrative and non-limiting value, where:

FIG. 1 is an exploded axonometric view of the disinfection/sterilization group according to the invention, showing an external unit, an internal unit and the disinfection/sterilization device according to the invention;

FIG. 1A, is a schematic sectional side view, of the disinfection/sterilization group according to the invention;

FIG. 2 is an axonometric top view of the disinfection/sterilization device according to the invention;

FIG. 3 is a bottom axonometric view of the disinfection/sterilization device according to the invention;

FIG. 4 is an axonometric view of first coupling means of a coupling group of the disinfection/sterilization device according to the invention;

FIG. 5 is a bottom axonometric view of disinfection/sterilization means of the disinfection/sterilization device according to the invention;

FIG. 6 is an axonometric view of the boxed longitudinal frame of the disinfection/sterilization device;

FIG. 6A, is a top view of the boxed longitudinal frame of the disinfection/sterilization device;

FIG. 6B, is a view of a detail of FIG. 6A.

### BEST MODE TO IMPLEMENT THE INVENTION

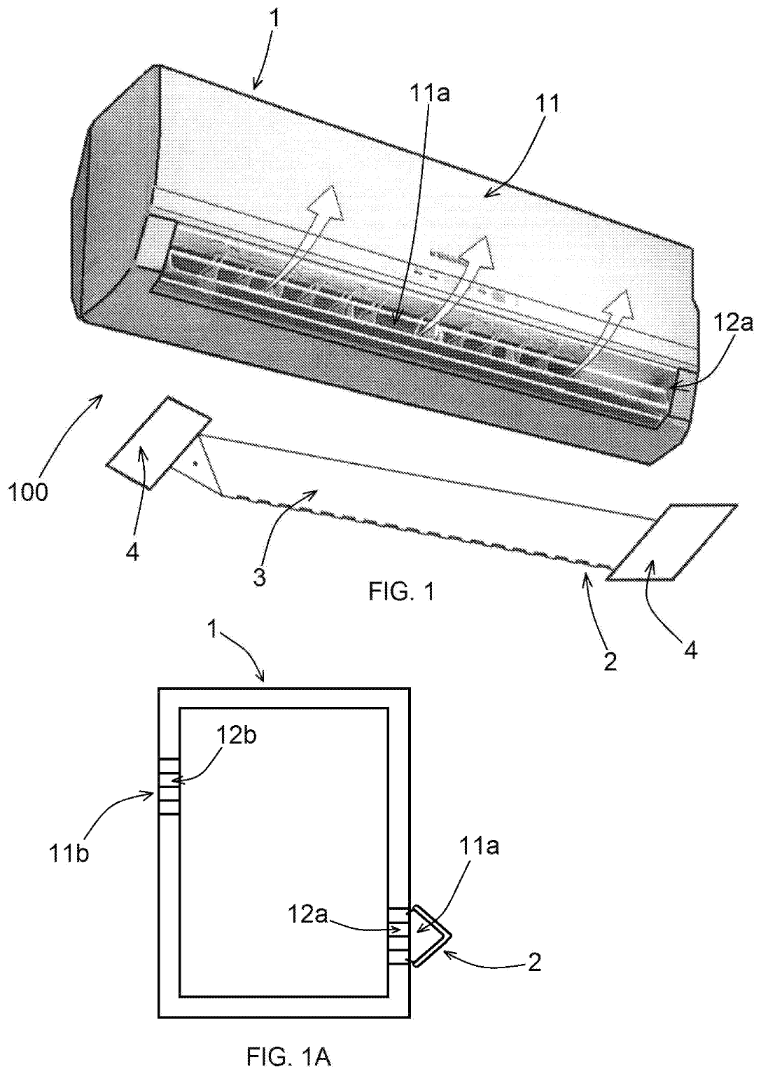

With reference to FIG. 1, the disinfection/sterilization group according to the invention is described, generally indicated with the reference number (100).

With reference to FIGS. 1 and 1A, the air disinfection/sterilization group (100) comprises an air conditioner (1), which provides hot or cold air in the environment in which it is installed and which comprises:
an internal unit (11), generally intended to be installed inside a room;
an external unit, generally intended to be installed outside said room;
fluid connection means, which connect the external unit to the internal unit (11);
electrical connection means, for the electrical connection between the external unit and the internal unit (11).

Specifically, the internal unit (11) comprises an outlet mouth (11a), crossed by air in output from the internal unit (11) and an inlet mouth (11b) crossed by air in input from the internal unit (11). The air conditioner (1) further comprises:
a grid (12a), applied in said outlet mouth (11a) and shaped so as to be crossed by air in output from the internal unit (11);
a grid (12b) applied in said inlet mouth (11b) and shaped so as to be crossed by air in input from the internal unit (11).

Usually, in common internal units, the aforementioned grids (12a, 12b) comprise a plurality of conventional adjustable lamellae as shown specifically in FIG. 1, for the grid (12a) applied to the outlet mouth (11a) of the internal unit (11).

However, the present invention is also suitable for those internal units (11) having grids (12a, 12b) of different structure.

The disinfection/sterilization group (100) further comprises a disinfection/sterilization device (2) capable of purifying/disinfecting/sterilizing the air immediately in output from the internal unit (11).

The description of the air disinfection/sterilization group (100) continues with reference to the grid (12a), applied to the outlet mouth (11a), for economy of description as shown in FIG. 1. Specifically, the disinfection/sterilization device (2) comprises:
a boxed longitudinal frame (3) located at the grid (12a) applied to the outlet mouth (11a) of the internal unit (11), extending longitudinally along the length of the grid (12a);
disinfection/sterilization means (30) adapted to purify the air in output from the grid (12a) and located inside said boxed longitudinal frame (3);
coupling group (4) which couples the boxed longitudinal frame (3) to the grid (12a) applied to the outlet mouth (11a).

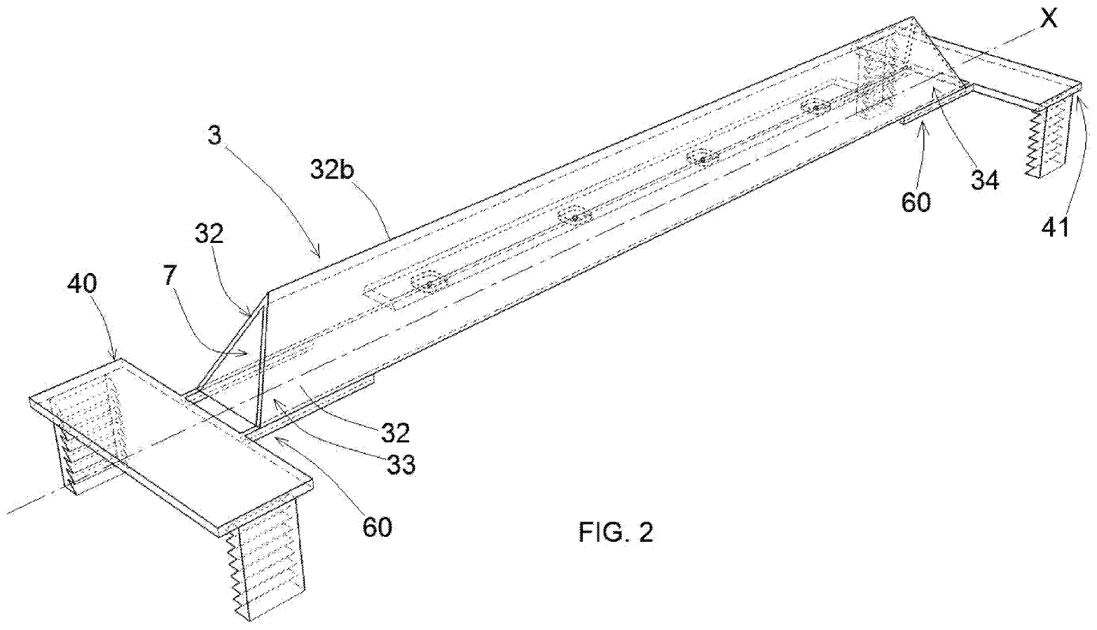

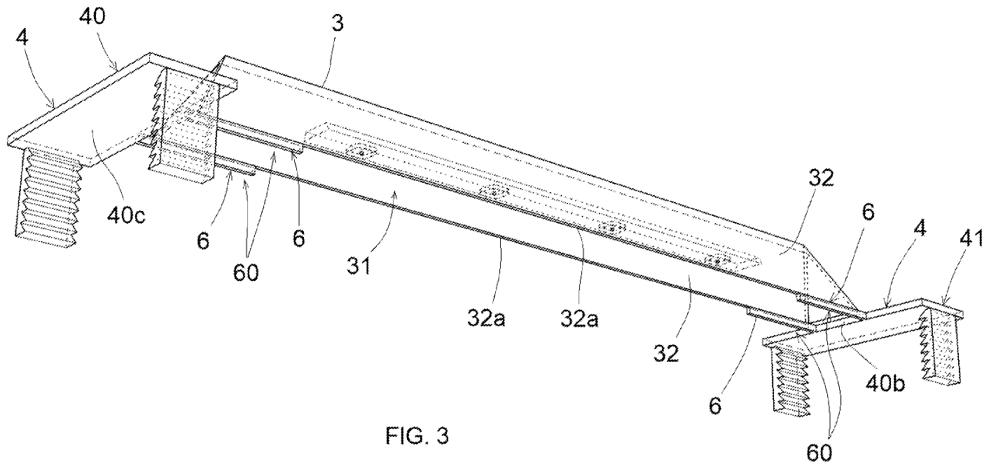

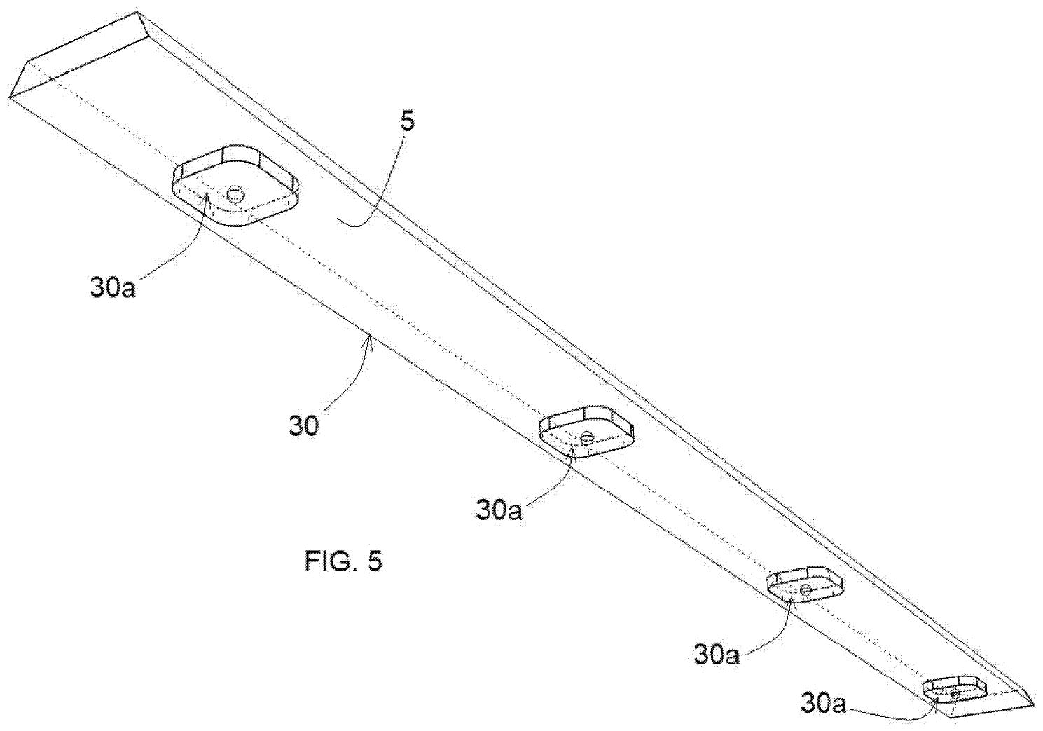

With reference to FIGS. 2 and 3, the boxed longitudinal frame (3) comprises:
a compartment (31) in which the disinfection/sterilization means (30) are housed, shown in FIG. 5;
two longitudinal walls (32), extending along a longitudinal axis (X), shown in FIG. 2, and each comprising a free edge (32a) and a connection edge (32b).

The two longitudinal walls (32) are connected to each other by means of the connection edges (32b) delimiting the compartment (31). Specifically and with reference to FIGS. 2, 3 and 6, the two longitudinal walls (32) are connected to each other forming a structure which, cross-sectioned, is triangular.

Precisely as shown in FIGS. 2, 3 and 6, the boxed longitudinal frame (3) thus has a substantially “V”-shaped profile defined by the two longitudinal walls (32).

Even if not shown in the accompanying figures, the disinfection/sterilization device (2) is shaped so that it can be inserted inside the air conditioner or any other type of air conditioning device, thanks to the shape and size of the boxed longitudinal frame (3).

In particular, in a different embodiment of the invention, the coupling group (4) can comprise a double-sided adhesive tape, arranged on one of the two side walls (32) of the boxed longitudinal frame (3), so as to be able to easily connect the latter if desired.

In particular, the free edge (32a) of the two longitudinal walls (32) faces the grid (12a), applied to the outlet mouth (11a) of the internal unit (11).

The aforementioned shape of the boxed longitudinal frame (3) favours the conveying of the air, coming from the grid (12a), into the compartment (31) where the disinfection/sterilization means (30) for sterilizing the air are specifically located.

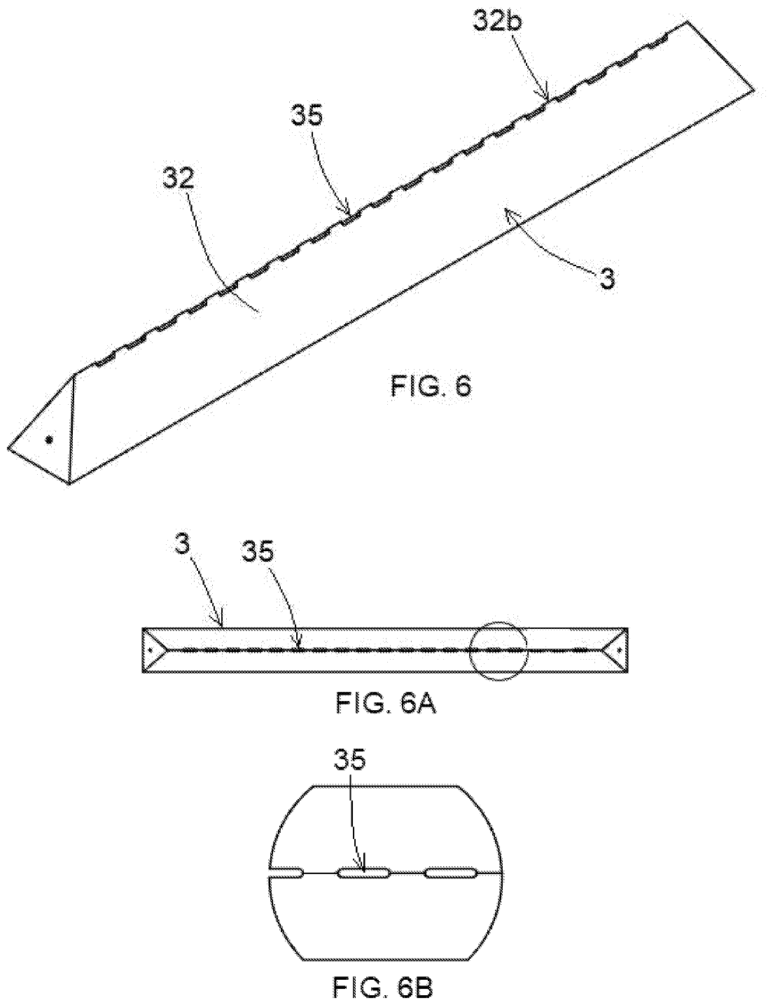

With reference to FIGS. 6, 6A and 6B, the boxed longitudinal frame (3) comprises elongated holes (35) at the connection edges (32b) of the two longitudinal walls (32); in other words, the holes (35) are obtained along the edge determined by the conjunction of the two longitudinal walls (32). Said holes (35) are intended to be crossed by the air purified by the disinfection/sterilization means (30) located in the compartment (30).

With reference to FIG. 5, the disinfection/sterilization means (30) comprise at least one or a plurality of LED lighting devices (represented as four by way of non-limiting example in the specific embodiment provided) of the UV-C type (30a), arranged on a longitudinal plate (5), which is inserted inside the compartment (31).

The four UV-C type LED lighting devices (30a) are facing the grid (12a), applied to the outlet mouth (11a) of the internal unit (11) and can each have a power, preferably of a value not less than 10 mW.

Said UV-C type LED lighting devices have a germicidal power on viruses and bacteria; in fact, such UV-C type LED lighting devices are capable of inactivating pathogens, inducing the breakage of the molecular bonds of the DNA and RNA chains contained in the bacteria and/or viruses present in the air.

Specifically, each UV-C type LED lighting device provides a small dose of energy density for a few seconds, which is sufficient to inactivate and inhibit the reproduction of a virus, when the virus is near the UV-C type LED lighting device.

Advantageously, the shape of the boxed longitudinal frame (3) is designed so as to ensure safety for the user, who must handle the disinfection/sterilization device or who is simply located near it. In fact, the shape of the boxed longitudinal frame (3) of the disinfection/sterilization device protects the user from any direct contact with the UV-C beam on the user's eyes and skin.

This protection is due to the fact that the disinfection/sterilization means are contained in the compartment (31) delimited by the two longitudinal walls (32), facing the air conditioner (1), so as to be able to shield the light rays emitted by the UV-C type LED lighting devices. Another benefit related to the shape of the boxed longitudinal frame (3) lies in the fact that the arrangement of the two side walls (32), manages to channel the emitted light in the best possible manner with a reduced amount of dispersions.

With reference to FIG. 2, the boxed longitudinal frame (3) comprises a first side end (33) and a second side end (34).

Always with reference to FIGS. 2 and 3, the coupling group (4) comprises:
first coupling means (40), located at the first side end (33), at the free edge (32a) of the two longitudinal walls (32);
second coupling means (41) located on the second side end (34), at the free edge (32a) of the two longitudinal walls (32).

The first coupling means (40) and the second coupling means (41) are equal and allow the boxed longitudinal frame (3) to be stably coupled to the grid (12a), applied to the outlet mouth (11a) of the internal unit (11).

Both the first coupling means (40) and the second coupling means (41) each comprise at least one serrated coupling portion (42), comprising a series of teeth (43) which interfere with the grid (12a) so that the first coupling means (40) and the second coupling means (41) cling to the grid (12a).

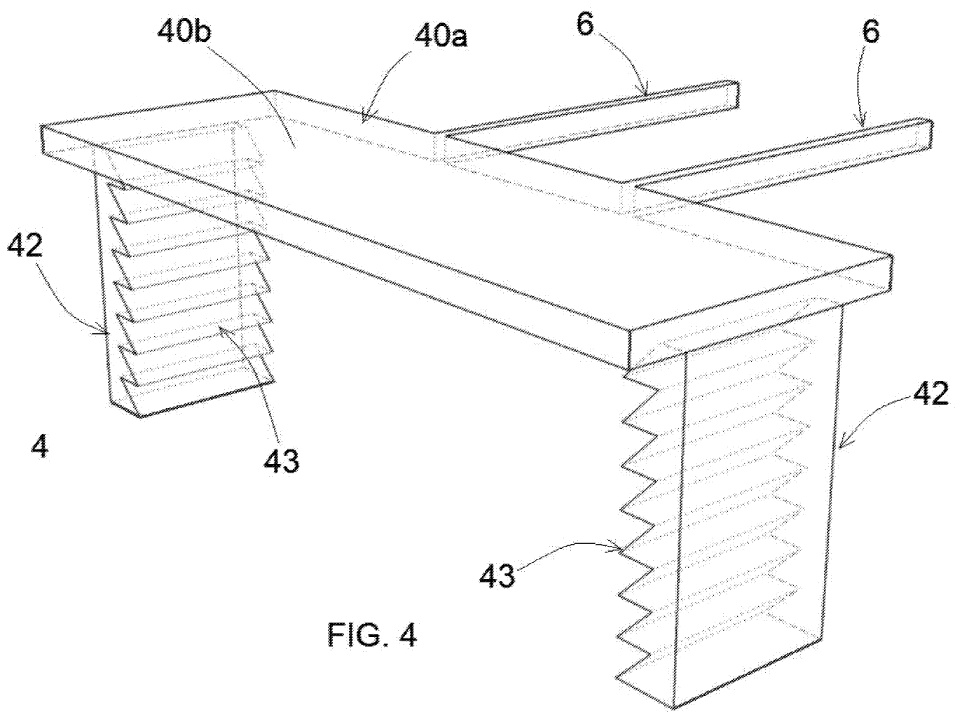

In the particular embodiment shown in the accompanying figures, said first coupling means (40) and said second coupling means (41) consist of a “C”-shaped bracket comprising:
a plate (40a), shown in FIG. 4 and comprising a first face (40b), a second face (40c) facing the grid (12a) and a side edge (40d) facing the boxed longitudinal frame (3);
two of the aforementioned serrated coupling portions (42), which are opposite and parallel to each other and which are connected to the plate (40a) defining the two parallel branches of the “C”-shaped bracket.

As mentioned above, each coupling portion (42) comprises the series of teeth (43) which is obtained on an internal face of the coupling portion (42), i.e., on the face facing the other coupling portion (42).

Therefore, to couple the first and the second coupling means (40, 41) to the grid (12a), it is necessary to bring the disinfection/sterilization device (2) closer to the outlet mouth of the internal unit (11) so that the coupling portions (42) partially penetrate the grid (12a) and their teeth interfere and grip the grid (12a) itself.

In particular, in the event in which the grid (11a) consists of the aforementioned adjustable lamellae, each coupling portion (42) is inserted between the space between two adjustable lamellae and the series of teeth (43) of the coupling portions (42) cling by interference to the external surfaces of the lamellae.

In other words, the serrated coupling portions (42), thanks to the series of teeth (43), cling to the grid (12a), or more precisely to the lamellae forming the aforementioned grid (12a).

Thanks to the series of teeth (43) it is also possible to more or less penetrate the coupling portions (43) in the grid, thereby adjusting the distance of the disinfection/sterilization device (2) with respect to the grid (12a), applied to the outlet mouth (11a) of the internal unit (11).

With reference to FIG. 2, in the preferred embodiment of the invention, the first and the second coupling means (40, 41) comprise translation means (60) shaped so as to translate the coupling means (40, 41) along the longitudinal axis (X) with respect to the boxed longitudinal frame (3). It is thereby possible to adjust the distance between the two coupling means (40, 41) and make the device versatile and adaptable to the width of the grid (12a) to which the device must be coupled. In the particular embodiment shown in the accompanying figures, the translation means (60) comprise two rods (6) parallel to each other which branch off from the side edge (40d) of the plate (40a) and which are slidably coupled with the free edges (32a) of the two longitudinal walls (32) of the boxed longitudinal frame (3).

In other words, at the first side end (33) of the boxed longitudinal frame (3), the free edges (32a) act as a guide for each rod (6) of the translation means (60) of the first coupling means (40) and, at the second side end (34) of the boxed longitudinal frame (3), the free edges (32a) act as a guide for each rod (6) of the translation means (60) of the second coupling means (41).

It should be noted that, although it is always shown in the accompanying figures that both the first and the second coupling means (40,41) can translate along the longitudinal axis (X), nothing would change in order to achieve the objectives pursued by the invention if only one of the two could translate while the other remained fixed. Thus, in this case, only one of the first and the second coupling means (40, 41) comprises the aforementioned translation means (60) while the other lacks them.

Although not shown in the accompanying figures, in a preferred embodiment of the invention said coupling portions (42) can be distanced or brought closer to each other so as to adapt to the shape of the grid.

In this case, the coupling portions (42) are connected to the plate (40a) in a movable manner by sliding means which allow one or both of the coupling portions (42) to slide along a transverse axis, preferably perpendicular to the longitudinal axis (X) of the boxed longitudinal frame (3). Thereby, the coupling portions (42) can couple to any type of grid (12a), making the disinfection/sterilization device (2) adapted to be coupled to any internal unit (11).

The boxed longitudinal frame (3) of the disinfection/sterilization device (2) of the disinfection/sterilization group (100) according to the invention finally comprises two closure caps (7), shown in FIG. 2, which laterally delimit said boxed longitudinal frame (3) near the first side end (33) and the second end (34).

Although not shown in the accompanying drawings, the disinfection/sterilization device (2) can also be coupled to the grid (12b), applied to the inlet mouth (11b) of the internal unit (11).

The disinfection/sterilization device (2) can also be used for other air conditioning systems, such as in ducted air conditioning systems.

The disinfection/sterilization device (2) can be coupled to ducted air conditioning systems by means of a grid or a delivery nozzle, so that it can be arranged inside the air conditioning system. In addition, the disinfection/sterilization device (2) can be installed near said grids by using glues or double-sided adhesive tapes.

Also in this case the disinfection/sterilization device (2) is capable of sterilizing and purifying the air immediately in output from a ducted air conditioning system.

Furthermore, the boxed longitudinal frame (3) of the disinfection/sterilization device (2) has a length of 20 cm and an operator can use and couple more than one disinfection/sterilization device (2) in series.

A further object of the present invention is a disinfection/sterilization device (2), as previously described, intended to be coupled to an air conditioning device.

In other words, although the disinfection/sterilization device (2) is preferably usable to be coupled with an internal unit (11) of an air conditioner (1), it can also be applied on other devices and air conditioning systems such as portable air conditioners, resistance fan heaters, air conditioning ducts and in similar situations having a grid on which the aforementioned disinfection/sterilization device (2) is coupled/installed. In addition, the disinfection/sterilization device (2) can be installed near said grids by using glues or double-sided adhesive tapes.

Also in this case the disinfection/sterilization device (2) is capable of sterilizing and purifying the air immediately in output from the air conditioning device.

The disinfection/sterilization device (2) is preferably made as the disinfection/sterilization device (2) forming part of the disinfection/sterilization group (100) described above, unless there are means for applying it in the various aforementioned situations, i.e., other air conditioning systems and plants, ducted or not.

With reference to FIGS. 1 and 1A, the disinfection/sterilization device (2) comprises:
a boxed longitudinal frame (3), which is intended to be located at an outlet mouth (11a) or an inlet mouth (11b) of an air conditioning device; said boxed longitudinal frame (3) extending longitudinally along the length of the outlet mouth (11a) or the inlet mouth (11b) of the air conditioning device;
disinfection/sterilization means (30), intended to purify air in output from the outlet mouth (11a) of the air conditioning device and/or in input from the inlet mouth (11b) of the air conditioning device;
   coupling group (4) or equivalent coupling means intended to couple said boxed longitudinal frame (3) to said outlet mouth (11a) or said inlet mouth (11b) of the air conditioning device or, alternatively, to couple said boxed longitudinal frame (3) to an air conditioning duct near said outlet mouth (11a) or said inlet mouth (11b) and, more generally, near the air intake and/or expulsion.

The description of the air disinfection/sterilization device (100) continues with reference to the grid (12a), applied to the outlet mouth (11a), for economy of description.

Referring to FIGS. 2 and 3, the boxed longitudinal frame (3) comprises:
a compartment (31) in which the disinfection/sterilization means (30) are housed, shown in FIG. 5;
two longitudinal walls (32), extending along a longitudinal axis (X), shown in FIG. 2, and each comprising a free edge (32a) and a connection edge (32b).

The two longitudinal walls (32) are connected to each other by means of the connection edges (32b) delimiting the compartment (31). Specifically and with reference to FIGS. 2, 3 and 6, the two longitudinal walls (32) are connected to each other forming a structure which, cross-sectioned, is triangular.

Precisely as shown in FIGS. 2, 3 and 6, the boxed longitudinal frame (3) thus has a substantially “V”-shaped profile defined by the two longitudinal walls (32).

In particular, the free edge (32a) of the two longitudinal walls (32) is intended to be facing the grid (12a) of the air conditioning device.

The aforementioned shape of the boxed longitudinal frame (3) is intended to favour the conveying of the air, coming from the grid (12a), into the compartment (31) where the disinfection/sterilization means (30) for sterilizing the air are specifically located.

With reference to FIGS. 6, 6A and 6B, the boxed longitudinal frame (3) comprises elongated holes (35) at the connection edges (32b) of the two longitudinal walls (32); in other words, the holes (35) are obtained along the edge determined by the conjunction of the two longitudinal walls (32). Said holes (35) favour the dispersion of the heat generated by the electronic components.

With reference to FIG. 5, the disinfection/sterilization means (30) comprise at least one or a plurality of LED lighting devices (represented as four by way of non-limiting example in the specific embodiment provided) of the UV-C type (30a); arranged on a longitudinal plate (5), which is inserted inside the compartment (31).

The four UV-C type LED lighting devices (30a) are intended to face the grid (11a) of the air conditioning device and can each have a power, preferably of a value not less than 10 mW.

Said UV-C type LED lighting devices have a germicidal power on viruses and bacteria; in fact, such UV-C type LED lighting devices are capable of inactivating pathogens, inducing the breakage of the molecular bonds of the DNA and RNA chains contained in the bacteria and/or viruses present in the air.

Specifically, each UV-C type LED lighting device provides a small dose of energy density for a few seconds, which is sufficient to inactivate and inhibit the reproduction of a virus, when the virus is near the UV-C type LED lighting device.

With reference to FIG. 2, the boxed longitudinal frame (3) comprises a first side end (33) and a second side end (34).

Always with reference to FIGS. 2 and 3, the coupling group (4) comprises:
first coupling means (40), located at the first side end (33), at the free edge (32a) of the two longitudinal walls (32);
second coupling means (41) located on the second side end (34), at the free edge (32a) of the two longitudinal walls (32).

The first coupling means (40) and the second coupling means (41) are equal and allow the boxed longitudinal frame (3) to be stably coupled to the grid (12a), applied to the outlet mouth (11a) of the air conditioning device.

Both the first coupling means (40) and the second coupling means (41) each comprise at least one serrated coupling portion (42), comprising a series of teeth (43) intended to interfere with the grid (12a) so that the first coupling means (40) and the second coupling means (41) cling to the grid (12a).

In the particular embodiment shown in the accompanying figures, said first coupling means (40) and said second coupling means (41) consist of a “C”-shaped bracket comprising:
a plate (40a), shown in FIG. 4 and comprising a first face (40b), a second face (40c) intended to face the grid (12a) and a side edge (40d) facing the boxed longitudinal frame (3);
two of the aforementioned serrated coupling portions (42), which are opposite and parallel to each other and which are connected to the plate (40a) defining the two parallel branches of the “C”-shaped bracket.

As mentioned above, each coupling portion (42) comprises the series of teeth (43) which is obtained on an internal face of the coupling portion (42), i.e., on the face facing the other coupling portion (42).

Therefore, in order to couple the first coupling means (40,41) to the grid (12a) of an air conditioning device, it is necessary to bring the disinfection/sterilization device (2) closer to the outlet mouth (11a) of the air conditioning device, so that the coupling portions (42) partially penetrate the grid (12a) and their teeth interfere and grip the grid (12a) itself.

In particular, in the event in which the grid (12a) consists of the aforementioned adjustable lamellae, each coupling portion (42) is intended to insert between the space between two adjustable lamellae and the series of teeth (43) of the coupling portions (42), clinging by interference to the external surfaces of the lamellae.

In other words, the serrated coupling portions (42), thanks to the series of teeth (43), are intended to cling to the grid (12a), or more precisely to the lamellae forming the aforementioned grid (12a). Thanks to the series of teeth (43) it is also possible to more or less penetrate the coupling portions (42) in the grid, thereby adjusting the distance of the disinfection/sterilization device (2) with respect to the grid (12a) of the air conditioning device.

In the preferred embodiment of the invention, the first and the second coupling means (40, 41) comprise translation means (60) shaped so as to translate the coupling means (40, 41) along the longitudinal axis (X) with respect to the boxed longitudinal frame (3). It is thereby possible to adjust the distance between the first and the second coupling means (40, 41) and to make the device versatile and adaptable to the width of the grid (12a) of the air conditioning device, to which the disinfection/sterilization device (2) is intended to be coupled.

In the particular embodiment shown in the accompanying figures, the translation means (60) comprise two rods (6) parallel to each other which branch off from the side edge (40d) of the plate (40a) and which are slidably coupled with the free edges (32a) of the two longitudinal walls (32) of the boxed longitudinal frame (3).

In other words, at the first side end (33) of the boxed longitudinal frame (3), the free edges (32a) act as a guide for each rod (6) of the translation means (60) of the first coupling means (40) and, at the second side end (34) of the boxed longitudinal frame (3), the free edges (32a) act as a guide for each rod (6) of the translation means (60) of the second coupling means (41).

It should be noted that, although it is always shown in the accompanying figures that both the first and the second coupling means (40,41) can translate along the longitudinal axis (X), nothing would change in order to achieve the objectives pursued by the invention if only one of the two could translate while the other is fixed. Thus, in this case, only one of said first and the second coupling means (40, 41) comprises the aforementioned translation means (60) while the other lacks them.

Although not shown in the accompanying figures, in a preferred embodiment of the invention said coupling portions (42) can be distanced or brought closer to each other so as to adapt to the shape of the grid.

In this case, the coupling portions (42) are connected to the plate (40a) in a movable manner by sliding means which allow one or both of the coupling portions (42) to slide along a transverse axis, preferably perpendicular to the longitudinal axis (X) of the boxed longitudinal frame (3). Thereby, the coupling portions (42) are intended to be able to couple to any type of grid (12a) of any type of air conditioning device, making the disinfection/sterilization device (2) adapted to be coupled to any air conditioning device.

The boxed longitudinal frame (3) of the disinfection/sterilization device (2) of the disinfection/sterilization group (100) according to the invention finally comprises two closure caps (7), shown in FIG. 2, which laterally delimit said boxed longitudinal frame (3) near the first side end (33) and the second end (34).

Although not shown in the accompanying figures, the disinfection/sterilization device (2) can also be coupled or installed with suitable fixing means near the grid (12a, 12b) or applied inside an air conditioning duct near the conditioned air intake and/or exhaust vents.

### INDUSTRIAL APPLICABILITY

The advantages of the disinfection/sterilization device (2) according to the invention are clear, which provides that it can be easily coupled/uncoupled to/from the internal unit of the air conditioner (1) or of another air conditioning system, so as to be able to easily and quickly perform a disinfection/sterilization of the air passing through the air conditioning device or system.

Another advantage of the present invention is that of envisaging that the disinfection/sterilization device (2) can be coupled or positioned/installed near a grid or inside an air conditioning ducting system, so as to immediately purify the air in output or in input from/in the grid itself or in the air conditioning device/system.

Finally, the shape of the first coupling means (40) and the second coupling means (41) envisages being able to adapt the disinfection/sterilization device (2) according to the invention to any type of air conditioner.

Numerous variations and modifications of details can be made to the present embodiment of the invention, within the ability of a person skilled in the art, which in any case fall within the scope of the invention expressed by the appended claims. Finally, the applicant reserves and claims the right to protection of the proposed patent, even if partially implemented.

1. Air disinfection/sterilization group, comprising:
an air conditioner comprising an external unit, an internal unit, fluid connection means connecting the external unit and the internal unit; the internal unit comprising an outlet mouth, crossed by air in output from the internal unit, and an inlet mouth, crossed by air in input from the internal unit; the air disinfection/sterilization group comprising a disinfection/sterilization device, comprising:
disinfection/sterilization means, adapted to purify air in output from at least one of the outlet mouth of the air conditioner and an input from the inlet mouth of the air conditioner;
a boxed longitudinal frame, which is located at an outlet mouth or an inlet mouth of the air conditioner the boxed longitudinal frame extending longitudinally along the length of the outlet mouth or the inlet mouth of the air conditioner, the boxed longitudinal frame comprising a compartment in which the disinfection/sterilization means are housed and comprising two longitudinal walls, which extend along a longitudinal axis (X) and each comprising a free edge and a connection edge; the two longitudinal walls being connected to each other by the connection edges delimiting the compartment, the two side walls being connected to each other forming a structure which, in cross-section, is triangular, each free edge facing at least one of the outlet mouth and the inlet mouth; and
means for coupling the boxed longitudinal frame to the air conditioner.

2. The air disinfection/sterilization group according to claim 1, wherein the disinfection/sterilization means comprises at least one IIV-C type LED.

3. The air disinfection/sterilization group according to claim 1, wherein the means for coupling the boxed longitudinal frame to the air conditioner comprises a coupling group which couples the boxed longitudinal frame to the inlet mouth or to the outlet mouth.

4. The air disinfection/sterilization group according to claim 3, wherein the boxed longitudinal frame comprises a first side end and a second side end; the coupling group comprising:
first coupling means, located on the first side end, at the free edge of the two longitudinal walls; and
second coupling means located on the second side end, at the free edge of the two longitudinal walls.

5. The air disinfection/sterilization group according to claim 4, wherein the at least one of the first and second coupling means comprises translation means shaped so as to make the at least one of the first and second coupling means translate along the longitudinal axis (X) with respect to the boxed longitudinal frame.

6. The air disinfection/sterilization group according to claim 4, wherein:
the air conditioner comprises a grid applied in the outlet mouth and shaped so as to be crossed by air in output from the internal unit;
the air conditioner comprises a grid applied in the inlet mouth and shaped so as to be crossed by air in input from the internal unit; and
the first coupling means and second coupling means each comprise at least one serrated coupling portion, comprising a series of teeth; the series of teeth interfering with at least one of the grids so that at least one of the first coupling means and the second coupling means remain anchored to the grid, applied to the outlet mouth, and to the grid, applied to the inlet mouth.

7. The air disinfection/sterilization group according to claim 6, wherein the first coupling means and second coupling means consist of a “C”-bracket comprising:
a plate;
two serrated coupling portions which are opposite and parallel to each other and which define the two parallel branches of the “C”-shaped bracket.

8. The air disinfection/sterilization group according to claim 1, wherein the means for coupling the boxed longitudinal frame to the air conditioner comprises gluing and adhesive tapes used to anchor the boxed longitudinal frame near the inlet mouth or the outlet mouth.

9. A disinfection/sterilization device for air conditioning systems, comprising:
disinfection/sterilization means, adapted to purify air in output from at least one of an outlet mouth of an air conditioning system and an input from an inlet mouth of the air conditioning system;
a boxed longitudinal frame located at the outlet mouth or the inlet mouth of an air conditioning system, the boxed longitudinal frame extending longitudinally along the length of the outlet mouth or the inlet mouth of the air conditioning systems, the boxed longitudinal frame comprising a compartment in which the disinfection/sterilization means are housed and comprising two longitudinal walls, extending along a longitudinal axis (X) and each comprising a free edge and a connection edge, the two longitudinal walls being connected to each other by the connection edges delimiting the compartment, the two side walls being connected to each other forming a structure which, in cross-section, is triangular, and each free edge being configured to face the outlet mouth or the inlet mouth of the air conditioning device;
means for coupling the boxed longitudinal frame with the air conditioning system.

10. The disinfection/sterilization device according to claim 9, wherein the disinfection/sterilization means comprises at least one IIV-C type LED.

11. The disinfection/sterilization device according to claim 9, wherein the means for coupling the boxed longitudinal frame with the air conditioning system comprise a coupling group which is configured to couple the boxed longitudinal frame to the outlet mouth or the inlet mouth of the air conditioning system.

12. The disinfection/sterilization device according to claim 9, wherein the boxed longitudinal frame comprises a first side end and a second side end, the coupling group comprising:
first coupling means, located on the first side end, at the free edge of the two longitudinal walls; and
second coupling means located on the second side end, at the free edge of the two longitudinal walls.

13. The disinfection/sterilization device according to claim 12, wherein the at least one of the first and second coupling means comprises translation means shaped so as to make the at least one of the first and second coupling means translate along the longitudinal axis (X) with respect to the boxed longitudinal frame.

14. The disinfection/sterilization device according to claim 12, wherein;
the air conditioning system comprises a grid, applied to the outlet mouth, and a grid applied to the inlet mouth; and
the first coupling means and second coupling means each comprise at least one serrated coupling portion, comprising a series of teeth; the series of teeth being configured to interfere with one of the grids so that at least one of the first coupling means and the second coupling means remain anchored to one of the grids.

15. The disinfection/sterilization device according to claim 14, wherein the first coupling means and second coupling means consist of a “C”-bracket comprising:
a plate; and
two serrated coupling portions which are opposite and parallel to each other and which define the two parallel branches of the “C”-shaped bracket.

16. The disinfection/sterilization device according to claim 9, wherein the means for coupling the boxed longitudinal frame to the air conditioning system comprise gluing and adhesive tapes used to anchor the boxed longitudinal frame near the inlet mouth or the outlet mouth.

17. The disinfection/sterilization device according to claim 9, wherein the means for coupling the boxed longitudinal frame to the air conditioning system comprises gluing and adhesive tapes used to anchor the boxed longitudinal frame to an air conditioning duct which is afferent to air intake or exhaust vents of an air conditioning system.

18. The air disinfection/sterilization group according to claim 5, wherein:
the air conditioner comprises a grid applied in the outlet mouth and shaped so as to be crossed by air in output from the internal unit;
the air conditioner comprises a grid applied in the inlet mouth and shaped so as to be crossed by air in input from the internal unit; and
the first coupling means and second coupling means each comprise at least one serrated coupling portion, comprising a series of teeth; the series of teeth interfering with at least one of the grids so that at least one of the first coupling means and the second coupling means remain anchored to the grid, applied to the outlet mouth, and to the grid, applied to the inlet mouth.

19. The disinfection/sterilization device according to claim 11, wherein the boxed longitudinal frame comprises a first side end and a second side end, the coupling group comprising:
first coupling means, located on the first side end, at the free edge of the two longitudinal walls; and
second coupling means located on the second side end, at the free edge of the two longitudinal walls.

20. The disinfection/sterilization device according to claim 13, wherein:
the air conditioning system comprises a grid, applied to the outlet mouth, and a grid applied to the inlet mouth; and
the first coupling means and second coupling means each comprise at least one serrated coupling portion, comprising a series of teeth; the series of teeth being configured to interfere with one of the grids so that at least one of the first coupling means and the second coupling means remain anchored to one of the grids.
